(19) United States
(12) Patent Application Publication
(10) Pub. No.: US 2012/0238881 A1
(43) Pub. Date: Sep. 20, 2012

(54) ORAL OPTICAL DIAGNOSING APPARATUS AND OPERATING METHOD THEREOF

(76) Inventors: Chung-Cheng CHOU, Taoyuan County (TW); William Wang, Taoyuan City (TW); Chung-Ping Chuang, Taoyuan City (TW)

(21) Appl. No.: 13/421,162

(22) Filed: Mar. 15, 2012

(30) Foreign Application Priority Data
Mar. 15, 2011 (TW) 100108756

(51) Int. Cl. A61B 6/00

(52) U.S. Cl. 600/476

(57) ABSTRACT

An oral optical diagnosing apparatus and operating method are disclosed. The oral optical diagnosing apparatus includes a positioning module, an optical sensing module, a processing module, and a display module. After the positioning module selects a region to be diagnosed in a mouth, the optical sensing module will perform optical sensing on the region to be diagnosed to obtain optical information related to the region to be diagnosed. The processing module is used to analyze the optical information to generate an optical diagnosed result. The display module is used to display the optical diagnosed result.

### BACKGROUND OF THE INVENTION

1. Field of the invention

The invention relates to optical diagnosing; in particular, to an oral optical diagnosing apparatus and operating method thereof capable of performing optical sensing to the surface and cross-section tissue state of the mouth in a non-invasive way, and generating early diagnosis result according to the sensed optical information to trace the patient's condition in a way of long-term and fixed point.

2. Description of the prior art

In recent years, with the continuous development of medical technology and biotechnology, the regions of medical diagnosing and biochemical test have become more and more important. Therefore, various instruments related to medical diagnosing and biochemical test are shown in the market. Especially, oral health is more and more important in the modern society, the oral diagnosing equipment used to diagnose the human oral state has been widely applied in the dental of hospitals and dental clinics.

According to the statistic data, in recent years, the ranking of mucositis is rising in Taiwan's cancer statistics list. It is because some people in Taiwan are addicted to eat areca. Conventionally, the mucositis is mostly visually observed by the doctor or in a biopsy way. In ordinary procedures, the patient will provide his/her disease condition to the doctor, and the doctor will visually observe the condition of the patient. If the doctor considers that the disease condition of the patient should be further confirmed, an oral sample biopsy procedure will be performed on the patient.

However, in the above-mentioned conventional oral diagnosing process, not only the diagnosing steps are complicated, but also the oral sample must be analyzed by other analyzing equipments. It is time-consuming and labor-intensive. In addition, the intrusion damage and uncomfortable inside the mouth of the patient are also drawbacks caused during the conventional oral diagnosing process.

Therefore, the invention provides an oral optical diagnosing apparatus and operating method thereof to solve the above-mentioned problems occurred in the prior arts.

### SUMMARY OF THE INVENTION

A first embodiment of the invention is an oral optical diagnosing apparatus. In this embodiment, the oral optical diagnosing apparatus includes a positioning module, an optical sensing module, a processing module, and a display module. The positioning module selects a region to be diagnosed in a mouth; the optical sensing module performs an optical sensing on the region to be diagnosed to obtain an optical information related to the region to be diagnosed; the processing module analyzes the optical information to generate an optical diagnosed result; the display module displays the optical diagnosed result.

In practical applications, the optical sensing module uses an optical interference technology (e.g., an optical coherence tomography (OCT) technology) to perform a longitudinal profile test on a mucosa under the region to be diagnosed in the mouth to obtain the optical information related to a longitudinal profile of the mucosa.

In addition, the optical sensing module includes an object lens and a temperature control unit disposed near the object lens. The temperature control unit performs a heated demisting process on the object lens to prevent the object lens from being disturbed by a mist in the mouth. The optical sensing module realizes a 3D motion in the mouth through motor components or in a manual way. The optical sensing module encased in a replaceable shell is disposed above a tongue in the mouth.

A second embodiment of the invention is an oral optical diagnosing apparatus operating method. The oral optical diagnosing apparatus includes a positioning module, an optical sensing module, a processing module, and a display module. The oral optical diagnosing apparatus operating method includes steps of: (a) the positioning module selecting a region to be diagnosed in a mouth; (b) the optical sensing module performing an optical sensing on the region to be diagnosed to obtain an optical information related to the region to be diagnosed; (c) the processing module analyzing the optical information to generate an optical diagnosed result; (d) the display module displaying the optical diagnosed result.

Compared to prior arts, the oral optical diagnosing apparatus and operating method thereof in the invention performs optical sensing to the surface and cross-section tissue state of the mouth in a non-invasive way, therefore, it not only can effectively improve the drawbacks of the intrusion damage and uncomfortable inside the mouth of the patient caused by the prior art, but also has no side effects caused by radioactive tests.

In addition, the oral optical diagnosing apparatus of the invention can generate early diagnosis result according to the sensed optical information related to the inner condition in the mouth, and provide the functions of determining potable target region and confirming the region to be diagnosed. Therefore, it can provide the patient functions of long-term and fixed point tracing disease conditions.

The advantage and spirit of the invention may be understood by the following detailed descriptions together with the appended drawings.

### BRIEF DESCRIPTION OF THE APPENDED DRAWINGS

FIG. 1 illustrates a function block diagram of the oral optical diagnosing apparatus in the first embodiment of the invention.

FIG. 2A illustrates a schematic diagram of the positioning module of the oral optical diagnosing apparatus including a micro-camera; FIG. 2B illustrates an image observed by the micro-camera.

FIG. 3A and FIG. 3B illustrate schematic diagrams of the optical sensing modules with the designs of single component type and two kits combination type respectively.

FIG. 4A and FIG. 4B illustrate schematic diagrams of the oral optical diagnosing apparatus reminding the operator to pay attention through lights when the distance between the oral optical diagnosing apparatus and the region to be diagnosed in the mouth is too small.

FIG. 5A and FIG. 5B illustrate schematic diagrams of the oral optical diagnosing apparatus designed in hand-held type or sensing base type according to different platform structures; FIG. 5C illustrates a schematic diagram of the auto scanning mechanism scans the entire region by moving a row of scanning units upward and downward (1D movement); FIG. 5D illustrates a schematic diagram of the auto scanning mechanism scans the entire region by moving a scanning unit upward, downward, leftward, and rightward (2D movement).

FIG. 6A illustrates a schematic diagram of the optical sensing module using motor component to realize 3D motion in patient's mouth; FIG. 6B and FIG. 6C illustrate the top view and the side view of the optical sensing module being encased in a replaceable shell and disposed above a tongue in the mouth.

FIG. 7 illustrates a flowchart of the oral optical diagnosing apparatus operating method in the second embodiment of the invention.

### DETAILED DESCRIPTION OF THE INVENTION

A scope of the invention is to provide an oral optical diagnosing apparatus and operating method thereof. The an oral optical diagnosing apparatus and operating method thereof performs optical sensing to the surface and cross-section tissue state of the mouth in a non-invasive way, and generates early diagnosis result according to the sensed optical information to provide the patient functions of long-term and fixed point tracing disease conditions.

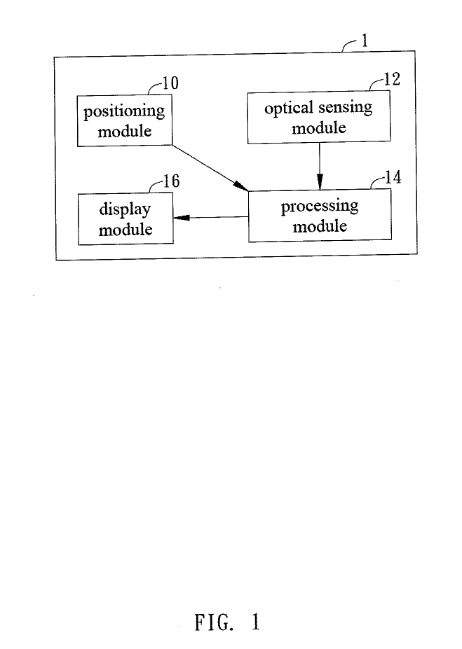

A first embodiment of the invention is an oral optical diagnosing apparatus. Please refer to FIG. 1. FIG. 1 illustrates a function block diagram of the oral optical diagnosing apparatus in the embodiment. As shown in FIG. 1, the oral optical diagnosing apparatus 1 includes a positioning module 10, an optical sensing module 12, a processing module 14, and a display module 16. Wherein, the processing module 14 is coupled to the positioning module 10; the processing module 14 is coupled to the optical sensing module 12; the display module 16 is coupled to the processing module 14. It should be noticed that the positioning module 10 can be not only coupled to the display module 16 through the processing module 14, but also directly coupled to the display module 16. That is to say, the display module 16 can be switched to display the surface (for confirming the diagnosed position) or observe practical diagnosed surface of depth without any specific limitations.

Next, the functions of the modules of the oral optical diagnosing apparatus 1 will be introduced respectively as follows.

At first, the diagnosed position of the patient should be continuously traced to obtain the change of its pathological state, therefore, the role that the positioning module 10 of the oral optical diagnosing apparatus 1 plays becomes very important. In this embodiment, the positioning module 10 firstly confirms the feature points in the patient's mouth to confirm that the patient's mouth is the optical diagnosed target of the oral optical diagnosing apparatus 1. After the patient's mouth is confirmed, the oral optical diagnosing apparatus 1 will set up the standard point in the patient's mouth, and select the region to be diagnosed in the patient's mouth through an image comparing method for the optical sensing module 12 to perform the following optical sensing procedure on the region to be diagnosed.

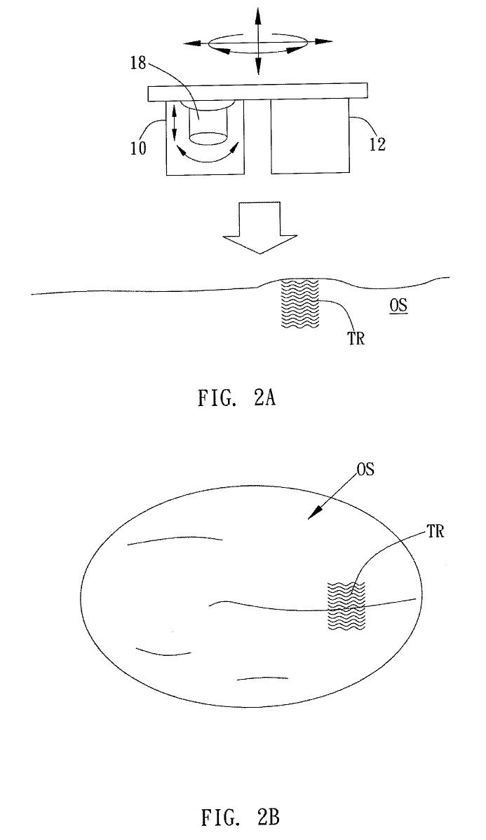

In practical applications, the positioning module 10 of the oral optical diagnosing apparatus 1 can have different types of design without any specific limitations. For example, as shown in FIG. 2A, the positioning module 10 can include a micro-camera 18 with functions of recording video and taking pictures. Because the lens of the micro-camera 18 can linearly move and rotate an angle, it can observe the oral tissue surface OS of the patient (the observed image is shown in FIG. 2B) to provide a reference when the positioning module 10 selects the region to be diagnosed TR from the oral tissue surface OS of the patient, but not limited to this.

Once the positioning module 10 selects the region to be diagnosed, the positioning module 10 can also capture many images of the region to be diagnosed and its neighboring regions through the micro-camera 18 for image comparing in the further. It should be noticed that the above-mentioned image comparing method can use not only the so-called “grayscale levels comparing method” to confirm the position of the region to be diagnosed through the grid distribution of levels, but also the so-called “eigenvalue comparing method” to use the special symbol target (e.g., the position of the teeth in the mouth) near the region to be diagnosed as important reference for comparing in the future.

That is to say, it is not necessary that the target compared when the positioning module 10 positions the region to be diagnosed is the region to be diagnosed. If the positioning module 10 can correctly position the region to be diagnosed by optical sensing, it can be other targets near the region to be diagnosed. Therefore, the target compared by the positioning module 10 when positioning is not necessary to be the same with the region to be diagnosed when the optical sensing module 12 performs optical sensing.

The optical sensing module 12 is used to perform optical sensing procedure on the region to be diagnosed to obtain the optical information related to the region to be diagnosed. In fact, the optical sensing module 12 uses an optical interference technology (e.g., an optical coherence tomography technology) to perform a longitudinal profile test on a mucosa under the region to be diagnosed in the mouth to obtain the optical information related to a longitudinal profile of the mucosa. In general, the depth that the optical sensing module 12 senses the tissue under the region to be diagnosed can be about 2˜3 mm, and the wavelength of the light it uses can be 1300 nm or 840 nm, but not limited to this case.

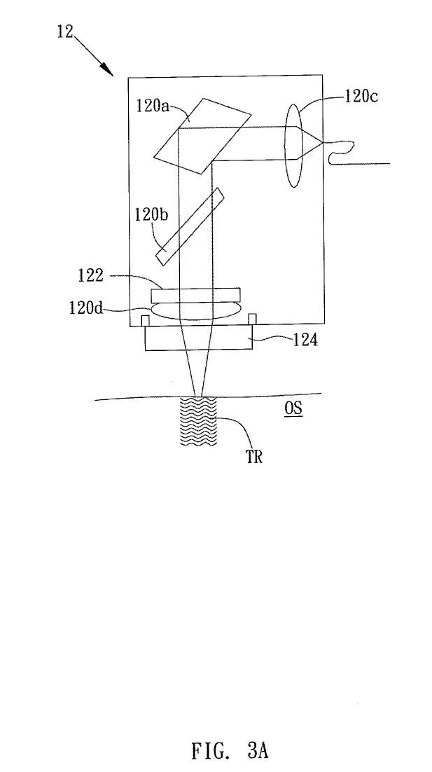

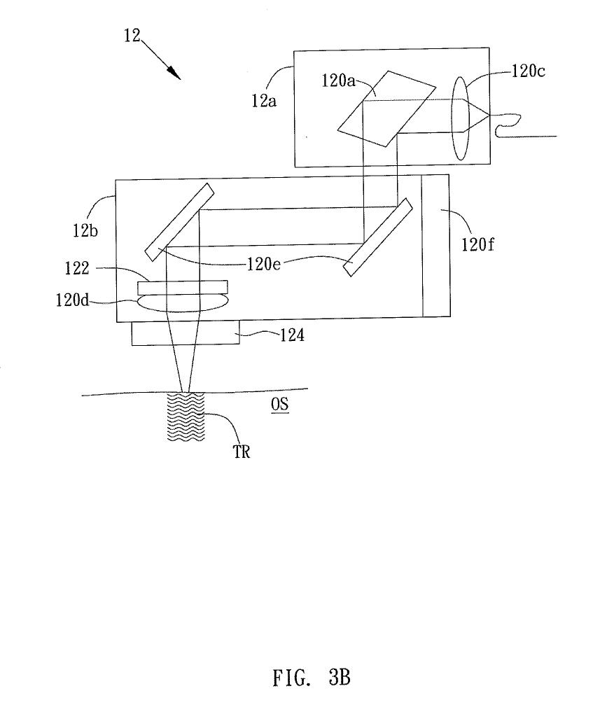

In practical applications, the structure of the optical sensing module 12 can be designed in different types. For example, please refer to FIG. 3A and FIG. 3B. FIG. 3A and FIG. 3B illustrate schematic diagrams of the optical sensing modules with the designs of single component type and two kits combination type respectively to achieve effects of flexibly designed, easy to upgrade, and easy to change the observing angle.

As shown in FIG. 3A, the optical sensing module 12 can include common optical components (including a rotation mirror 120a, a light splitter 120b, a collimating lens 120c, and an objective lens 120d), a temperature control unit 122, and a contact end replacing component 124. It should be noticed that the main function of the light splitter 120b is for the user to use the reflected light of the oral tissue surface OS cooperated with the built-in image sensor (not shown in figures, such as CCD or CMOS type of image sensor) to observe the region to be diagnosed TR on the oral tissue surface OS, but not limited to this. That is to say, the design of the optical sensing module 12 in FIG. 3A can be integrated with the positioning module 10.

As to the optical sensing module 12 in FIG. 3B, it is formed by the combination of two kits 12a and 12b. The kit 12a includes the rotation mirror 120a and the collimating lens 120c, and the kit 12b includes the objective lens 120d, a reflector 120e, the temperature control unit 122, and the contact end replacing component 124. It should be noticed that the optical sensing module 12 in FIG. 3B has no light splitter 120b and built-in image sensor, therefore, the optical sensing module 12 in FIG. 3B fails to be integrated with the positioning module 10. In fact, the image sensor 120f (CCD or CMOS type of image sensor) can be plugged in the optical sensing module 12 in FIG. 3B, but not limited to this.

From above, it can be known that the design type of the optical sensing module of the oral optical diagnosing apparatus 1 has no specific limitations. It can choose to use different designs of single component type or combination type, or integrated with the positioning module. In addition, the two reflectors 120e in FIG. 3B can be mirrors, and the distance between the two reflectors 120e has no fixed limitations, and it can be adjusted based on practical needs.

It should be noticed that in order to prevent the objective lens 120d from being interfered by the mist in the mouth when the optical sensing module 12 performs optical sensing on the region to be diagnosed, as shown in FIG. 3A and FIG. 3B, the temperature control unit 122 will be disposed near the objective lens 120d to perform the heating and mist removing procedure to the objective lens 120d. In fact, the temperature control unit 122 can be a heating ring, but not limited to this.

As to the contact end replacing component 124 in FIG. 3A and FIG. 3B, it can be designed to be detachable type, disposable type, or release paper tear-off type to prevent the surface of the contact end of the optical sensing module 12 from being contaminated, and the usage of the optical sensing module 12 can be personalized.

In practical applications, the positioning module 10 of the invention can provide various aids for different disease backgrounds of the patients. For example, the initial wound or ulcer may be formed on the region to be diagnosed in patient's mouth, therefore, when the oral optical diagnosing apparatus 1 performs optical sensing, direct contact between the oral optical diagnosing apparatus 1 and the region to be diagnosed on the oral tissue surface should be avoided, so that the patient will feel more comfortable. In view of this, the positioning module 10 of the oral optical diagnosing apparatus 1 can further provide the distance measuring function which is realized by comparing the sizes of surface pattern or directly by the built-in optical transceiver (not shown in figures).

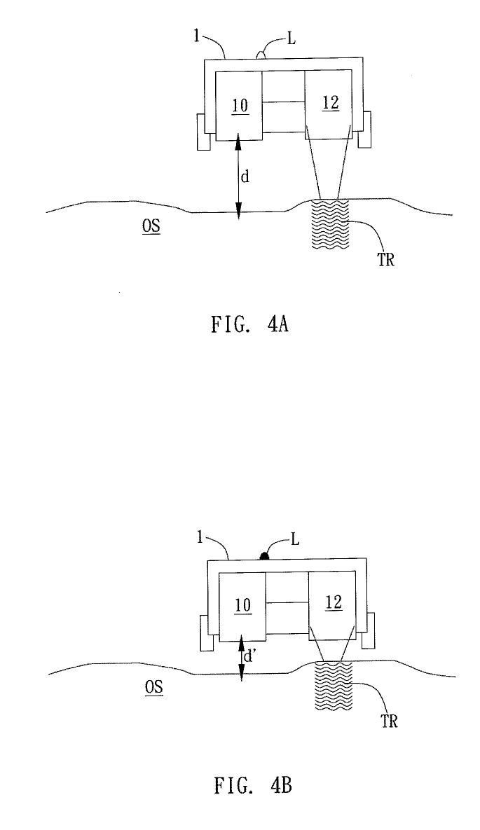

As shown in FIG. 4A, the shortest distanced d between the oral optical diagnosing apparatus 1 and the region to be diagnosed TR of the oral tissue surface OS measured by the positioning module 10 of the oral optical diagnosing apparatus 1 is not small enough, therefore, the warning light L of the oral optical diagnosing apparatus 1 will not emit lights. However, as shown in FIG. 4B, when the shortest distanced d′ between the oral optical diagnosing apparatus 1 and the region to be diagnosed TR of the oral tissue surface OS measured by the positioning module 10 of the oral optical diagnosing apparatus 1 is too small, the warning light L of the oral optical diagnosing apparatus 1 will emit lights or make sounds to remind the user that the movement of the oral optical diagnosing apparatus 1 should be carefully controlled not to contact with the region to be diagnosed TR.

The optical information captured by the optical sensing module 12 can be transmitted to the processing module 14 through the light path (such as fiber or photoconductive element). Then, the processing module 14 will process the received optical information and perform longitudinal profile analysis on the optical information to generate an optical diagnosed result. At last, the display module 16 will display the optical diagnosed result for the user to observe it.

In fact, the way that the display module 16 displays the optical diagnosed result has no specific limitations. For example, the display module 16 can display the optical diagnosed result through images with different colors or shades; the display module 16 can display the optical diagnosed result through the voices with different volumes, frequencies, tempos; the display module 16 can display the optical diagnosed result through different temperatures; the display module 16 can also emit lights with different brightness or colors to display the optical diagnosed result.

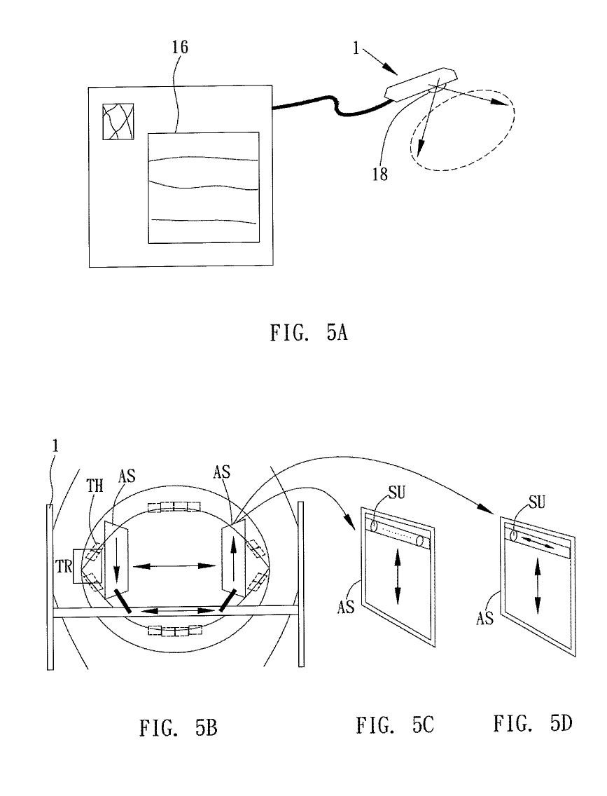

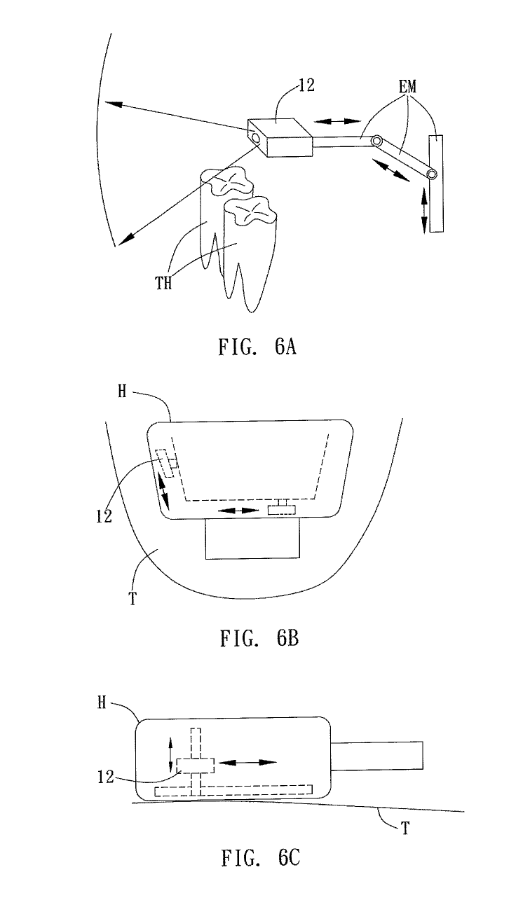

In practical applications, the oral optical diagnosing apparatus 1 can be reasonably configured according to different platform structures, for example, designs of hand-held type (FIG. 5A) or sensing base type (FIG. 5B). As shown in FIG. 5A, if the oral optical diagnosing apparatus 1 uses the design of hand-held type, it can directly use the micro-camera 18 cooperated with the image analysis software to achieve the effects of contact-free and accurate positioning.

As shown in FIG. 5B, if the oral optical diagnosing apparatus 1 uses the design of sensing base type, it can use default optical machine structure cooperated with the feature values (e.g., the related position of the tooth TH and the region to be diagnosed TR) as the reference for scanning to position the region to be diagnosed TR. And, the oral optical diagnosing apparatus 1 can also directly use the auto scanning mechanism AS to scan the entire region; therefore, it is not necessary to perform the procedure of manual determining by eyes.

FIG. 5C illustrates a schematic diagram of the auto scanning mechanism AS scans the entire region by moving a row of scanning units SU upward and downward (1D movement); FIG. 5D illustrates a schematic diagram of the auto scanning mechanism AS scans the entire region by moving a scanning unit SU upward, downward, leftward, and rightward (2D movement).

In practical applications, the design of sensing base used in the above-mentioned oral optical diagnosing apparatus 1 can be used cooperated with the optical sensing module 12 with different designs. For example, as shown in FIG. 6A, the optical sensing module 12 can realize a 3D motion in the mouth through motor components EM or in a manual way; as shown in FIG. 6B, the optical sensing module 21 encased in a replaceable shell H is disposed above a tongue T in the mouth to provide a more comfortable and clean diagnosis environment for the patients.

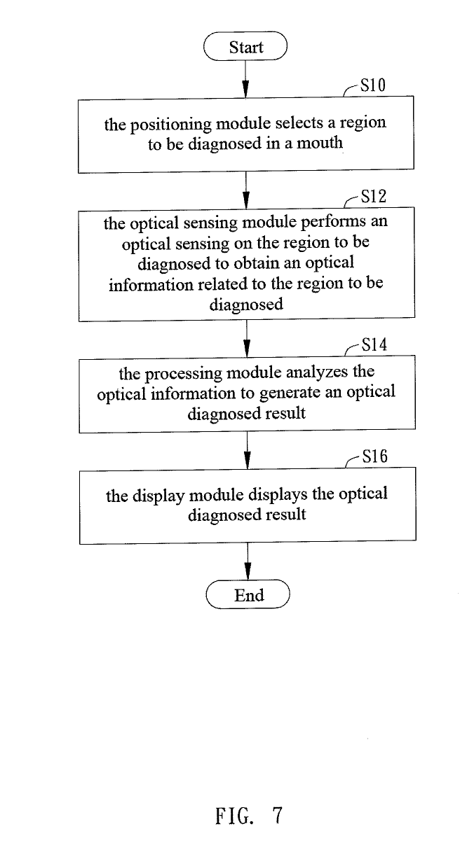

A second embodiment of the invention is an oral optical diagnosing apparatus operating method. In this embodiment, the oral optical diagnosing apparatus includes a positioning module, an optical sensing module, a processing module, and a display module. Please refer to FIG. 7. FIG. 7 illustrates a flowchart of the oral optical diagnosing apparatus operating method in this embodiment.

As shown in FIG. 7, at first, the method performs step S10 that the positioning module selects a region to be diagnosed in a mouth. In fact, in step S10, the positioning module will firstly confirm the feature points in the patient's mouth to confirm that the patient's mouth is the optical diagnosed target of the oral optical diagnosing apparatus. After the patient's mouth is confirmed, the oral optical diagnosing apparatus will set up the standard point in the patient's mouth, and select the region to be diagnosed in the patient's mouth through an image comparing method or a feature point comparing method. It is not necessary that the target compared when the positioning module positions the region to be diagnosed is the region to be diagnosed. If the positioning module can correctly position the region to be diagnosed by optical sensing, it can be other targets near the region to be diagnosed. Therefore, the target compared by the positioning module when positioning is not necessary to be the same with the region to be diagnosed when the optical sensing module performs optical sensing.

After the region to be diagnosed is selected, the method performs step S12 that the optical sensing module performs an optical sensing on the region to be diagnosed to obtain an optical information related to the region to be diagnosed. In fact, the optical sensing module uses an optical interference technology (e.g., an optical coherence tomography technology) to perform a longitudinal profile test on a mucosa under the region to be diagnosed in the mouth to obtain the optical information related to a longitudinal profile of the mucosa. In general, the depth that the optical sensing module senses the tissue under the region to be diagnosed can be about 2˜3 mm, and the wavelength of the light it uses can be 1300 nm or 840 nm, but not limited to this case.

In addition, the optical sensing module includes an object lens and a temperature control unit. The temperature control unit can be a heating ring disposed near the object lens, but not limited to this. In step S12, when the optical sensing module performs optical sensing to the region to the diagnosed, the temperature control unit will perform a heated demisting process on the object lens to prevent the object lens from being disturbed by a mist in the mouth.

In practical applications, the optical sensing module can realize a 3D motion in the mouth through motor components or in a manual way, and the optical sensing module encased in a replaceable shell is disposed above a tongue in the mouth to provide a more comfortable and clean environment for the patients.

Then, the method performs step S14 that the processing module analyzes the optical information to generate an optical diagnosed result. In fact, the processing module can compare the optical diagnosed result with the previous optical diagnosed result to obtain early diagnosis result related to the inner condition in the mouth. By doing so, the oral optical diagnosing apparatus operating method of the invention does not have the complicated procedures of the visual observation by doctor or sample biopsy in the prior art.

At last, the method will perform step S16 that the display module displays the optical diagnosed result for the user or doctor to observe it to judge the condition of the patient. In fact, the way that the display module displays the optical diagnosed result in step S16 has no specific limitations. For example, the display module can display the optical diagnosed result through images with different colors or shades, the voices with different volumes, frequencies, tempos, different temperatures, and different brightness or colors.

Compared to prior arts, the oral optical diagnosing apparatus and operating method thereof in the invention performs optical sensing to the surface and cross-section tissue state of the mouth in a non-invasive way, therefore, it not only can effectively improve the drawbacks of the intrusion damage and uncomfortable inside the mouth of the patient caused by the prior art, but also has no side effects caused by radioactive tests.

In addition, the oral optical diagnosing apparatus of the invention can generate early diagnosis result according to the sensed optical information related to the inner condition in the mouth, and provide the functions of determining potable target region and confirming the region to be diagnosed. Therefore, it can provide the patient functions of long-term and fixed point tracing disease conditions.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An oral optical diagnosing apparatus, comprising:
a positioning module, for selecting a region to be diagnosed in a mouth;
an optical sensing module, coupled to the positioning module, the optical sensing module performing an optical sensing on the region to be diagnosed to obtain an optical information related to the region to be diagnosed;
a processing module, coupled to the optical sensing module, the processing module analyzing the optical information to generate an optical diagnosed result; and
a display module, coupled to the processing module, for displaying the optical diagnosed result.

2. The oral optical diagnosing apparatus of claim 1, wherein the optical sensing module uses an optical interference technology to perform a longitudinal profile test on a mucosa under the region to be diagnosed in the mouth to obtain the optical information related to a longitudinal profile of the mucosa.

3. The oral optical diagnosing apparatus of claim 1, wherein the optical sensing module comprises:
an object lens; and
a temperature control unit, disposed near the object lens, for performing a heated demisting process on the object lens to prevent the object lens from being disturbed by a mist in the mouth.

4. The oral optical diagnosing apparatus of claim 1, wherein the optical sensing module realizes a 3D motion in the mouth through motor components or in a manual way.

5. The oral optical diagnosing apparatus of claim 1, wherein the optical sensing module encased in a replaceable shell is disposed above a tongue in the mouth.

6. The oral optical diagnosing apparatus of claim 1, wherein the optical sensing module is integrated with the positioning module to observe the region to be diagnosed through a light splitter cooperated with a built-in image sensor.

7. A method of operating an oral an oral optical diagnosing apparatus, the oral optical diagnosing apparatus comprising a positioning module, an optical sensing module, a processing module, and a display module, the method comprising steps of
(a) the positioning module selecting a region to be diagnosed in a mouth;
(b) the optical sensing module performing an optical sensing on the region to be diagnosed to obtain an optical information related to the region to be diagnosed;
(c) the processing module analyzing the optical information to generate an optical diagnosed result; and
(d) the display module displaying the optical diagnosed result.

8. The method of claim 7, wherein in step (b), the optical sensing module uses an optical interference technology to perform a longitudinal profile test on a mucosa under the region to be diagnosed in the mouth to obtain the optical information related to a longitudinal profile of the mucosa.

9. The method of claim 7, wherein the optical sensing module comprises an object lens and a temperature control unit disposed near the object lens, the temperature control unit performs a heated demisting process on the object lens to prevent the object lens from being disturbed by a mist in the mouth.

10. The method of claim 7, wherein the optical sensing module realizes a 3D motion in the mouth through motor components or in a manual way.

11. The method of claim 7, wherein the optical sensing module encased in a replaceable shell is disposed above a tongue in the mouth.

12. The method of claim 7, wherein the optical sensing module is integrated with the positioning module to observe the region to be diagnosed through a light splitter cooperated with a built-in image sensor.
